(19) United States
(12) Patent Application Publication
(10) Pub. No.: US 2026/0027049 A1
(43) Pub. Date: Jan. 29, 2026

(54) AVACOPAN FORMULATIONS

(71) Applicant: AMGEN INC., THOUSAND OAKS, CA (US)

(72) Inventors: Bhanu Chander BEJGUM, NEWBURY PARK, CA (US); Yuan-Hon KIANG, NEWBURY PARK, CA (US); Fernando Antonio ALVAREZ-NUNEZ, NEWBURY PARK, CA (US); Rajneet OBEROI, OAK PARK, CA (US); Fatima Ahmad BARBAR-SMILEY, DUBLIN, OH (US); Stephanie Mabel KONG, WALTHAM, MA (US); Rafael SARABIA, LOS ALTOS, CA (US)

(21) Appl. No.: 19/276,811

(22) Filed: Jul. 22, 2025

Related U.S. Application Data
(60) Provisional application No. 63/674,654, filed on Jul. 23, 2024.
(60) Provisional application No. 63/807,025, filed on May 16, 2025.

(51) Int. Cl. A61K 9/08; A61K 9/00; A61K 31/451; A61K 31/675; A61K 39/00; A61K 47/14; C07K 16/28

(52) U.S. Cl. CPC A61K 9/08; A61K 9/0053; A61K 31/451; A61K 31/675; A61K 47/14; C07K 16/2887; A61K 2039/545

(57) ABSTRACT

The disclosure provides formulations comprising avocapan and one or more medium-chain triglycerides.

### CROSS-REFERENCES TO RELATED APPLICATIONS

This application claims the benefit of priority to U.S. Provisional Patent Application No. 63/674,654, filed Jul. 23, 2024, and U.S. Provisional Patent Application No. 63/807,025, filed May 16, 2025.

### FIELD

The present invention is directed to formulations of (2R,3S)-2-(4-(cyclopentylamino)phenyl)-1-(2-fluoro-6-methylbenzoyl)-N-(4-methyl-3-(trifluoromethyl)phenyl) piperidine-3-carboxamide (hereinafter referred to as avacopan or a compound of Formula I) and methods of administering the same.

### BACKGROUND

The complement system plays a central role in the clearance of immune complexes and in immune responses to infectious agents, foreign antigens, virus infected cells and tumor cells. Inappropriate or excessive activation of the complement system can lead to harmful, and even potentially life-threatening consequences due to severe inflammation and resulting tissue destruction. These consequences are clinically manifested in various disorders including septic shock; myocardial, as well as intestinal ischemia/reperfusion injury; graft rejection; organ failure; nephritis; pathological inflammation; and autoimmune diseases.

The complement system is composed of a group of proteins that are normally present in the serum in an inactive state. Activation of the complement system encompasses mainly three distinct pathways, i.e., the classical, the alternative, and the lectin pathway (V. M. Holers, In Clinical Immunology: Principles and Practice, ed. R. R. Rich, Mosby Press; 1996, 363-391): 1) The classical pathway is a calcium/magnesium-dependent cascade, which is normally activated by the formation of antigen-antibody complexes. It can also be activated in an antibody-independent manner by the binding of C-reactive protein, complexed with ligand, and by many pathogens including gram-negative bacteria. 2) The alternative pathway is a magnesium-dependent cascade which is activated by deposition and activation of complement component C3 on certain susceptible surfaces (e.g. cell wall polysaccharides of yeast and bacteria, and certain biopolymer materials). 3) The lectin pathway involves the initial binding of mannose-binding lectin and the subsequent activation of complement components C2 and C4, which are common to the classical complement pathway (Matsushita, M. et al., J. Exp. Med. 176:1497-1502 (1992); Suankratay, C. et al., J. Immunol. 160:3006-3013 (1998)).

The activation of the complement pathway generates biologically active fragments of complement proteins, e.g. C3a, C4a and C5a anaphylatoxins and C5b-9 membrane attack complexes (MAC), all which mediate inflammatory responses by affecting leukocyte chemotaxis; activating macrophages, neutrophils, platelets, mast cells and endothelial cells; and increasing vascular permeability, cytolysis and tissue injury.

Complement component C5a is one of the most potent proinflammatory mediators of the complement system. The anaphylactic C5a peptide is 100 times more potent, on a molar basis, in eliciting inflammatory responses than C3a. C5a is the activated form of C5 (190 kD, molecular weight). C5a is present in human serum at approximately 80 μg/ml (Kohler, P. F. et al., J. Immunol. 99:1211-1216 (1967)). It is composed of two polypeptide chains, α and β, with approximate molecular weights of 115 kD and 75 kD, respectively (Tack, B. F. et al., Biochemistry 18:1490-1497 (1979)). Biosynthesized as a single-chain promolecule, C5 is enzymatically cleaved into a two-chain structure during processing and secretion. After cleavage, the two chains are held together by at least one disulphide bond as well as noncovalent interactions (Ooi, Y. M. et al., J. Immunol. 124:2494-2498 (1980)). Avacopan, having Formula I

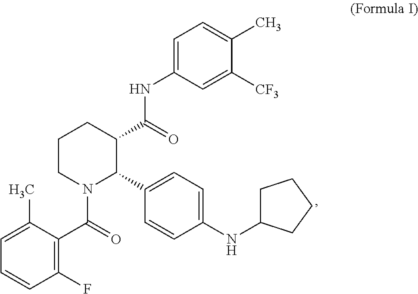 [SMILES: Cc1ccc(NC(=O)[C@H]2CCCN(C(=O)c3c(C)cccc3F)[C@H]2c2ccc(NC3CCCC3)cc2)cc1C(F)(F)F]

is useful for treating C5aR mediated diseases. For example, avacopan is useful for the treatment of inflammatory diseases, including treatment of anti-neutrophil cytoplasmic autoantibody-associated vasculitis (also referred to as ANCA-associated vasculitis or AAV), including granulomatosis with polyangiitis (GPA) and microscopic polyangiitis (MPA), complement 3 glomerulopathy (C3G), hidradenitis suppurativa (HS), or lupus nephritis, or any combination of the foregoing. United States Patent Application Publication Number 2016/0229802 A1, published on Aug. 17, 2016, discloses avacopan.

Avacopan is classed as a compound belonging to Class II of the Biopharmaceutics Classification System (BCS) having poor solubility in the aqueous environment of the gastrointestinal (GI) tract but high permeability across membranes. Thus, its absorption is controlled by its solubility and rate of dissolution in the GI tract.

Oral delivery of poorly water-soluble drugs for pediatric patients is expected to remain challenging for the foreseeable future (See page 20 of in S. Salunke et al., Advanced Drug Delivery Reviews 190 (2022) 114507). Pediatric formulations that were developed for adult formulations for poorly soluble drug substances include the following as described in S. Salunke et al:

Pediatric formulations of drug substances that leverage the solubility-enhancing technology used in the adult formulation:
Lopinavir/Ritonavir-oral pellets in capsule
Ivacaftor-Oral granules in packet containing solid dispersion
Lumacaftor/Ivacaftor-Oral granules in packet containing solid dispersion
Amprenavir-Oral solution containing vitamin E polyethylene glycol succinate, polyethylene glycol, and propylene glycol
Cyclosporin A-Oral solution containing ethanol, mono-ditriglycerides, polyoxyl 40 hydrogenated castor oil, and propylene glycol
Aprepitant-powder for oral suspension containing nanocrystalline API

Pediatric formulations reformulated as oral liquids:
Ritonavir-Oral solution containing ethanol, water, polyoxyl 35 castor oil, and propylene glycol
Lopinavir/Ritonavir-Oral solution containing ethanol and propylene glycol
Tipranavir-Oral solution containing polyethylene glycol, vitamin E polyethylene glycol succinate, water, and propylene glycol

Accordingly, there remains a need for a suitable formulation for avacopan that can be administered to patients in need thereof, particularly patients that have difficulty swallowing and/or pediatric patients. The present disclosure addresses these needs and provides related advantages as well.

### SUMMARY

Disclosed are formulations of avacopan. In another embodiment of this disclosure relates to non-aqueous liquid formulations of avacopan. Another embodiment of this disclosure relates to formulations of avacopan comprising a solution of avacopan in one or more medium-chain triglycerides. In other embodiments of this disclosure, the formulations of avacopan of this disclosure is manufactured by dissolving either crystalline or spray dried amorphous avacopan in a non-aqueous liquid such as one or more medium-chain triglycerides. United States Patent Application Publication Number 2021/0137907 published on May 13, 2021, discloses a free base crystalline form of avacopan. United States Patent Application Publication Number 2021/0139426 A1 published on May 13, 2021, discloses an amorphous form of avacopan.

In other embodiments of this disclosure, the formulations of avacopan of this disclosure are suitable for oral administration to pediatric patients. Other embodiments of this disclosure are method of treating a disease mediated by C5aR in a subject in need of treatment, the method comprising administering to the subject a therapeutically effective amount of the formulations described herein.

### BRIEF DESCRIPTION OF THE FIGURES

FIG. 1 is a process flow diagram for the manufacture of a formulation comprising avacopan and Labrafac Lipophile WL 1349TM (Labrafac™).

### DETAILED DESCRIPTION

The present disclosure is based, in part, on the discovery that the formulations as disclosed herein comprising avacopan and certain excipients in certain amounts result in formulations suitable for oral administration to patients that have difficulty swallowing or pediatric patients.

### Definitions

The following definitions are provided to assist in understanding the scope of this disclosure. Unless defined otherwise, all technical and scientific terms used herein have the same meaning as is commonly understood by one of ordinary skill in the art to which this disclosure belongs.

The term “a disease mediated by C5aR” means inflammatory disorders and autoimmune disorders associated with the complement system and particular involving C5a and its receptor C5aR. C5aR is expressed on a broad spectrum of immune and non-immune cells and are involved in cellular functions and physiological processes during homeostasis and inflammation. Dysregulated C5a-mediated inflammation contributes to diseases such as anti-neutrophil cytoplasmic autoantibody-associated vasculitis (also referred to as ANCA-associated vasculitis or AAV), including granulomatosis with polyangiitis (GPA) and microscopic polyangiitis (MPA), complement 3 glomerulopathy (C3G), hidradenitis suppurativa (HS), and lupus nephritis, and others. Avacopan received FDA approval as an adjunctive treatment of adult patients with severe active anti-neutrophil cytoplasmic autoantibody (ANCA)-associated vasculitis (granulomatosis with polyangiitis [GPA] and microscopic polyangiitis [MPA]) in combination with standard therapy including glucocorticoids. Treatment of C3G patients with avacopan in a Phase 2 Accolade clinical trial demonstrated statistically significant improvement in renal function as measured by eGFR compared to placebo over 26 weeks of blinded treatment. Treatment of HS patients with Compound in a Phase 3 Aurora clinical trial demonstrated statistically significant dose-dependent improvement in HISCR (Hidradenitis Suppurativa Clinical Response) vs. placebo in pre-specified Hurley Stage III (severe HS) patients at 12 weeks.

The term “patient” or “subject” refers to humans and other mammals. The term “mammal” as used herein includes, for example, humans, non-human primates, cattle, sheep, goats, pigs, horses, cats, dog, rabbits, rodents (e.g., rats or mice), and monkeys. Human subjects include neonates, infants, juveniles, adults, and geriatric subjects.

The terms “pediatric patient” or “pediatric subject” refers to a human from 6 years of age to under 18 years of age. Pediatric patients include child patients and adolescent patients.

The term “child patient” or “child subject” refers to a human from 6 years of age to under 12 years of age.

The term “adolescent patient” refers to a human from 12 years of age to under 18 years of age.

The term “pharmaceutically acceptable” refers to a species or component that is generally safe, non-toxic, and neither biologically nor otherwise undesirable for use in a subject.

The term “pharmaceutically acceptable salt” refers to a salt of a compound that possesses the desired pharmacological activity of the parent compound and that is not biologically or otherwise undesirable for its end use. Pharmaceutically acceptable salts include, for example, acid addition salts formed with inorganic acids (e.g., hydrochloric acid, hydrobromic acid, sulfuric acid, nitric acid, phosphoric acid) or formed with organic acids (e.g., acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid). Pharmaceutically acceptable salts also include, for example, salts formed when an acidic proton present in the parent compound either is replaced by a metal ion (e.g., an alkali metal ion, an alkaline earth ion, or an aluminum ion) or associates with an organic base (e.g., ethanolamine, diethanolamine, triethanolamine, N-methylglucamine, dicyclohexylamine). Additionally, the salts of the compounds described herein, can exist in either hydrated or anhydrous form or as solvates with other solvent molecules.

The term “therapeutically effective amount” as used herein refers to that amount of a compound disclosed herein that elicits a desired biological or medical response in a cell, a tissue, a system, or a subject.

The term “medium-chain triglycerides (MCT)” refers to a class of lipids in which three intermediate carbon length saturated fats ranging from 6 to 12 carbons in length

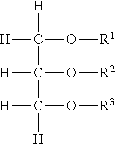

wherein R¹, R² and R³ is

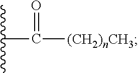

and each n is independently selected from 6-12. In other embodiments, each n is independently selected from 6-10. In other embodiments, each n is independently 6 or 8.

Medium-chain triglycerides are available commercially and they can also be manufactrued from various sources including coconut oil, certain kernal oils (such as palm kernal oil and babassa oil), and various animal products such as milk fat.

### Formulations

The formulation of this disclosure comprises avacopan and medium-chain triglycerides that serves as a vehicle and a solubilizer.

### Medium-Chain Triglycerides:

The vehicle and solubilizer for the formulations of this disclosure comprises one or more medium-chain triglycerides. Non-limiting examples of medium-chain triglycerides include medium chain triglycerides of caprylic (also known as octanoyl or 8 carbon fatty acid) and capric (also known as decanoyl or 10 carbon fatty acid) acids. Other nonlimiting examples of medium-chain triglycerides include Miglyol® 812 N, Miglyol® 810 N, Witarix® 60/40 MCT, Labrafac™ Lipophile WL 1349, Captex® Medium-Chain Triglycerides, and Crodamol GTCC-PN.

Labrafac™ contains mostly caprylic/capric triglycerides with greater than 95% of the fatty acid distribution being 8 carbon (caprylic acid) and 10 carbon (capric acid). Labrafac™ is also described as glycerides, mixed decanoyl and octanoyl. The full distribution of fatty acids in Labrafac™ has been described as less than or equal to 2% C6 (6 carbon) fatty acids, 50-80% C8 (8 carbon) fatty acids, 20-50% C10 (10 carbon) fatty acids, less than or equal to 3% C12 (12 carbon) fatty acids, less than or equal to 1% C14 (14 carbon) fatty acids, and less than or equal to C16 (16 carbon) fatty acids. The sum of caprylic and capric fatty acids in Labrafac™ is greater than or equal to 95%. Labrafac™ can be made by an esterification reaction between glycerol and the relevant fatty acids, such as caprylic and capric acids.

### Composition Containers

In one embodiment, the formulation of this disclosure is in a container, wherein the formulation is 3 mg/mL of avacopan in one or more medium-chain triglycerides as described in this disclosure. In another embodiment, the formulation of this disclosure is contained in a 120 mL container, wherein the formulation is 3 mg/mL of avacopan in one or more medium-chain triglycerides as described in this disclosure. In another embodiment, the formulation of this disclosure is contained in a 120 mL container, wherein the formulation is 3 mg/mL of avacopan in one or more medium-chain triglycerides containing mostly caprylic/capric triglycerides with greater than 95% of the fatty acid distribution being 8 carbon (caprylic acid) and 10 carbon (capric acid). In another embodiment, the formulation of this disclosure is contained in a 120 mL container, wherein the formulation is 3 mg/ml of avacopan in one or more medium-chain triglycerides containing less than 2% C6 (6 carbon) fatty acids, 50-80% C8 (8 carbon) fatty acids, 20-50% C10 (10 carbon) fatty acids, less than 3% C12 (12 carbon) fatty acids, and less than 1% C14 (14 carbon) fatty acids. In other embodiments, the formulation of this disclosure is contained in multi-dose stock-keeping unit (SKU), in 150 mL Type III amber glass bottles with 130 mL fill volume (120 mL label volume+10 mL necessary excess volume), optionally with 28 mm polypropylene child resistant closures with polypropylene liners . . .

Although the foregoing invention has been described in some detail by way of illustration and example for purposes of clarity of understanding, it will be evident that certain changes and modifications may be practiced within the scope of the appended claims.

### Embodiments

Embodiment 1 of this disclosure relates to a formulation comprising avacopan, or a pharmaceutically acceptable salt thereof, and one or more medium-chain triglycerides.

Embodiment 2 of this disclosure relates to the formulation of Embodiment 1, wherein the one or more medium-chain triglyceride are triglycerides of saturated fatty acids that are from 6 to 12 carbons in length. The triglycerides of Embodiment 2 may also have a saturated fatty acids of 14 or 16 carbons in smaller percentages such as 2% or less of the total fatty acid content.

Embodiment 3 of this disclosure relates to the formulation of Embodiment 1 or 2, wherein the one or more medium-chain triglycerides comprises triglycerides of caprylic and capric acids.

Embodiment 4 of this disclosure relates to the formulation of Embodiment 3, wherein the one or more medium-chain triglycerides are triglycerides of about 50% to about 80% capric acid and from 20% to about 50% caprylic acid.

Embodiment 5 of this disclosure relates to the formulation of Embodiment 4, wherein the one or more medium-chain triglycerides aretriglycerides of at least about 95% of caprylic and capric acid.

Embodiment 6 of this disclosure relates to the formulation of any one of

Embodiments 1-5, comprising avacopan in an amount of 0.1-1.0% (w/w).

Embodiment 7 of this disclosure relates to the formulation of Embodiment 6, comprising avacopan in an amount of 0.2, 0.3%, 0.4%, 0.5%, or 0.6% (w/w).

Embodiment 7 (a) of this disclosure relates to the formulation of Embodiment 7, comprising avacopan in an amount of 0.2, 0.3%, or 0.4% (w/w).

Embodiment 8 of this disclosure relates to the formulation of Embodiment 6, comprising avacopan in an amount of 0.3% (w/w).

Embodiment 9 of this disclosure relates to the formulation of any one of Embodiments 1-5, comprising avacopan in an amount of 3 mg/ml.

Embodiment 10 of this disclosure relates to the formulation of Embodiment 6, comprising avacopan in an amount of 0.6% (w/w).

Embodiment 11 of this disclosure relates to the formulation of any one of Embodiments 1-5, comprising avacopan in an amount of 6 mg/ml.

Embodiment 12 of this disclosure relates to the formulation of any one of Embodiments 1-5, comprising avacopan in an amount of 0.3% (w/w) and one or more medium chain triglycerides in an amount of 99.7% (w/w).

Embodiment 13 of this disclosure relates to the formulation of any one of Embodiments 1-5, comprising avacopan in an amount of 0.6% (w/w) and one or more medium chain triglycerides in an amount of 99.4% (w/w).

Embodiment 14 of this disclosure relates to the formulation of any one of Embodiments 1-13, wherein the formulation if suitable for oral administration.

Embodiment 15 of this disclosure relates to the formulation of any one of Embodiments 1-14, wherein the formulation is suitable for oral administration to patients who have difficulty swallowing or pediatric patients.

Embodiment 15 (a) of this disclosure relates to the formulation of Embodiment 15, wherein the formulation is suitable for oral administration to patients that have difficulty swallowing.

Embodiment 15 (b) of this disclosure relates to the formulation of Embodiment 15, wherein the formulation is suitable for oral administration to pediatric patients.

Embodiment 16 of this disclosure relates to the formulation of any one of Embodiments 1-15, wherein the formulation is an immediate release formulation.

Embodiment 17 of this disclosure relates to the formulation of any one of Embodiments 1-15, wherein the formulation prior to administration is contained in glass or plastic containers with child-resistant closures.

Embodiment 18 of this disclosure relates to the formulation of Embodiment 17, wherein the child-resistant closure comprises polypropylene liners.

Embodiment 19 of this disclosure relates to the formulation of any one of Embodiments 1-18 for use as a medicament.

Embodiment 20 of this disclosure relates to the formulation of any one of Embodiments 1-19, wherein avacopan is in a free base form.

Embodiment 21 of this disclosure relates to the formulation of any one of Embodiments 1-20, wherein the formulation is a liquid formulation.

Embodiment 22 of this disclosure relates to the formulation of any one of Embodiments 1-21, wherein the formulation does not comprise an antimicrobial agent.

Embodiment 23 of this disclosure relates to the method of treating a disease mediated by C5aR in a subject in need of treatment, the method comprising administering to the subject a therapeutically effective amount of the formulation of any one of Embodiments 1-22.

Embodiment 24 of this disclosure relates to the method of Embodiment 23, wherein the disease mediated by C5aR is anti-neutrophil cytoplasmic autoantibody-associated vasculitis, complement 3 glomerulopathy, hidradenitis suppurativa, or lupus nephritis, or any combination of the foregoing.

Embodiment 25 of this disclosure relates to the method of Embodiment 24, wherein the disease mediated by C5aR is anti-neutrophil cytoplasmic autoantibody-associated vasculitis.

Embodiment 26 of this disclosure relates to the method of any one of Embodiments 22-25, wherein the subject receives of avacopan in one or more medium-chain triglyceride selected from one of the following doses: 9 mg BID; 15 mg BID; and 30 mg BID.

Embodiment 26 (a) of this disclosure relates to Embodiment 26, wherein the subject is from age 6 to <12 years of age and receives 3 mg/mL of avacopan in one or more medium-chain triglyceride selected from one of the following doses:
9 mg (3 mL) BID for subjects ≥15 kg to <20 kg;
15 mg (5 mL) BID for subjects ≥20 kg to <50 kg; and
30 mg (10 mL) BID for subjects ≥50 kg.

Embodiment 26 (b) of this disclosure relates to Embodiment 26, wherein the subject is from age 12 to <18 years of age and receives 3 mg/mL of avacopan in one or more medium-chain triglyceride selected from one of the following doses: 15 mg (5 mL) BID; and 30 mg (10 mL) BID.

Embodiment 26 (c) of this disclosure relates to Embodiment 26 (b), wherein the subject is from age 12 to <18 years of age and receives 3 mg/mL of avacopan in one or more medium-chain triglyceride selected from one of the following doses:
15 mg (5 mL) BID for subjects ≥20 kg to <50 kg; and
30 mg (10 mL) BID for subjects ≥50 kg.

Embodiment 27 of this disclosure relates to the method of Embodiment 26, wherein the subject receives 3 mg/mL of avacopan in one or more medium-chain triglyceride at a dose of 9 mg BID.

Embodiment 27 (a) of this disclosure relates to the method of Embodiment 27, wherein the subject receives 3 mg/mL of avacopan in one or more medium-chain triglyceride at a dose of 3 mL BID.

Embodiment 28 of this disclosure relates to the method of Embodiment 26, wherein the subject receives 3 mg/mL of avacopan in one or more medium-chain triglyceride at a dose of 15 mg BID.

Embodiment 28 (a) of this disclosure relates to the method of Embodiment 26, wherein the subject receives 3 mg/mL of avacopan in one or more medium-chain triglyceride at a dose of 5 mL BID.

Embodiment 29 of this disclosure relates to the method of Embodiment 26, wherein the subject receives 3 mg/mL of avacopan in one or more medium-chain triglyceride at a dose of 30 mg BID.

Embodiment 29 (a) of this disclosure relates to the method of Embodiment 26, wherein the subject receives 3 mg/mL of avacopan in one or more medium-chain triglyceride at a dose of 10 mL BID.

Embodiment 30 of this disclosure relates to the method of Embodiment 27, wherein the subject is ≥15 kg to <20 kg.

Embodiment 31 of this disclosure relates to the method of Embodiment 28, wherein the subject is ≥20 kg to <50 kg.

Embodiment 32 of this disclosure relates to the method of any one of

Embodiments 26-32, wherein the subject is ≥50 kg.

Embodiment 33 of this disclosure relates to the method of any one of

Embodiments 26-32, wherein the subject is a patient that has difficulty swallowing solids or is a pediatric patient.

Embodiment 33 (a) of this disclosure relates to Embodiment 33, wherein the patient has difficulty swallowing solids.

Embodiment 33 (b) of this disclosure relates to the method of any one of Embodiments22, wherein the subject is a patient is a pediatric patient.

Embodiment 34 of this disclosure relates to the method of any one of Embodiments 23-33, wherein the subject is administered rituximab or cyclophosphamide in combination with the formulation.

Embodiment 35 of this disclosure relates to the method of Embodiment 34, wherein cyclophosphamide is administered to the subject in an amount of 15 mg/kg every 2 weeks for the first 3 doses, then every 3 weeks for the next 3 to 6 doses.

Embodiment 36 of this disclosure relates to the method of Embodiment 34, wherein cyclophosphamide is administered to the subject in an amount of 750 mg/m² every 4 weeks for 4 to 7 doses.

Embodiment 37 of this disclosure relates to the method of Embodiment 34, wherein rituximab is administered to the subject in an amount of 375 mg/m² in 4 doses given one week apart.

Embodiment 38 of this disclosure relates to the method of Embodiment 34, wherein rituximab is administered to the subject in an amount of 750 mg/m² in 2 doses given 2 weeks apart.

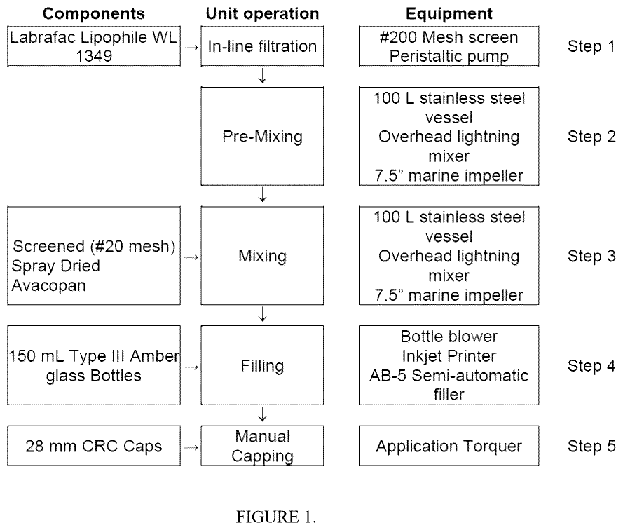

Embodiment 39 of this disclosure relates to a process of making a formulation comprising: providing crystalline or spray-dried amorphous avocapan; and
dissolving the crystalline or spray-dried amorphous avacopan in one or more medium-chain triglycerides, wherein the one or more medium-chain triglyceride are triglycerides of saturated fatty acids that are from 6 to 12 carbons in length. FIG. 1 depicts just one non-limiting example of this process that may be suitable for larger batches. In other embodiments, the free base form of avacopan is used when providing the crystalline or spray-dried amorphous avacopan.

Embodiment 39 (a) of this disclosure relates to the process of Embodiment 34, wherein crystalline avacopan is dissolved in one or more medium-chain triglycerides. In other embodiments, the free base form of avacopan is used when providing the crystalline avacopan.

Embodiment 39 (b) of this disclosure relates to the process of Embodiment 34, wherein spray-dried amorphous avacopan is dissolved in one or more medium-chain triglycerides. In other embodiments, the free base form of avacopan is used when providing the spray-dried amorphous avacopan.

Embodiment 40 of this disclosure relates to the process of Embodiment 39, wherein the one or more medium-chain triglyceride are triglycerides of caprylic and capric acids.

Embodiment 40 (a) of this disclosure relates to the formulation of Embodiment 40, wherein the one or more medium-chain triglyceride comprises from about 50% to about 80% capric acid and from 20% to about 50% caprylic acid.

Embodiment 40 (b) of this disclosure relates to the formulation of Embodiment 40, wherein the one or more medium-chain triglyceride comprises triglycerides with 95% of caprylic and capric acids.

Embodiment 41 of this disclosure relates to a formulation of avacopan made by the process of Embodiment 39 or 40.

Embodiment 42 of this disclosure relates to the formulation of Embodiment 41, comprising avacopan in an amount of 0.1-1.0% (w/w).

Embodiment 43 of this disclosure relates to the formulation of Embodiment 42, comprising avacopan in an amount of 0.3% (w/w).

Embodiment 44 of this disclosure relates to the formulation of claim 43, comprising avacopan in an amount of 3 mg/ml.

Embodiment 45 of this disclosure relates to the formulation of any one of Embodiments 41-44, wherein the formulation is in liquid form.

Other embodiments relate to alternative dosing regiments for remission-maintenance treatment of pediatric antineutrophil cytoplasmic antibody-associated-vasculitis (azathioprine.

[AZA] or methotrexate [MTX] versus rituximab [RTX].
AZA regimen
   2 to 3 mg/kg orally daily, maximum 200 mg/day
MTX regimen
   0.5 to 0.7 mg/kg (maximum 25 mg) once weekly
   subcutaneous recommended over oral therapy
   should not be used in renal failure
   AZA and MTX considered equivalent for RTX comparison
RTX regimen 1
   375 mg/m² IV (maximum 500 mg) at day 0 and 14, every 6 months
RTX regimen 2
   750 mg/m² IV (maximum 1,000 mg) at day 0, every 6 months
   maximum individual dose of 1,000 mg
RTX regimens 1 and 2
   are 2-dose (day 0 and 14) and 1-dose (day 0)
   every 6 months dosing schedules adapted from adult practice and considered equivalent

For either RTX regimen 1 or 2, repeat dosing every 6 months regardless of B cell population (tracked with CD19 cell count)·
When to start remission-maintenance treatment:
   Following remission-induction with CYC
      begin maintenance within 1 month of completing CYC therapy
   Following remission-induction with RTX
      if using AZA or MTX for maintenance, begin 3 months after RTX if using RTX for maintenance, begin 6 months after RTX induction

### EXAMPLES

### Example 1-Formulation Manufacturing

Avacopan formulation (3 mg/mL) is manufactured by dissolving spray-dried avacopan in Labrafac™ via mixing using an overhead mixer. Alternatively, crystalline avacopan can also be used in the manufacture of the avacopan formulation, but the process would be expected to take longer. The bulk is filled into the primary containers using a semi-automatic pump. The flow diagram for the pivotal scale (50 L) manufacturing process is provided in FIG. 1. The formulation that is manufactured is contained in a 120 mL container that can then be used for oral administration to subjects. The manufacturing steps are described below.

1. Dispense Labrafac™ into a 100L stainless steel vessel following in-line filtration through a #200 mesh screen using a peristaltic pump.

2. Pre-mix the Labrafac™ in Step 1 using a lightning mixer equipped with 7.5″ marine impeller.

3. Add prescreened (#20 mesh) spray dried avacopan to the pre-mixed Labrafac™ in step 2 and mix using the 7.5″ marine impeller until complete dissolution of DS.

4. Fill the bulk solution from Step 3 to target fill weight into previously cleaned and labeled 150 cc glass bottles using AB-5 semi-automatic filler and record the fill weight of each bottle.

5. Manually place the cap to the filled glass bottles and apply target application torque.

### Spray-Dried Avacopan Manufacturing Description

A feed solution of avacopan is prepared by dissolving the solid crystalline avacopan in acetone to afford a stock solution. The solution is then charged into the spray-dryer through an in-line filter apparatus. A high-pressure pump is used to feed the solution to the nozzle and to atomize into a spray. The atomized solution is dried in a drying chamber by co-current hot nitrogen gas. The stream containing the wet spray-dried product leaves the drying chamber and enters the cyclone where the solids are separated and collected in the collection bag. The spray dried drug substance (avacopan) is collected and dried.

### Example 2-Avacopan Solution Formulation Screening

A set of trials were designed for various avacopan solutions with different excipients by performing stress studies over a period of a period of a week. The following formulations in Tables 1 and 2 were tested.

TABLE 1
Compositions for Avacopan Solution Formulations 6 mg/mL

| | 1A | | 1B | | 2A | | 2B | |
|---|---|---|---|---|---|---|---|---|
| | | g/per | | g/per | | g/per | | g/per |
| | % | 50 mL | % | 50 mL | % | 50 mL | % | 50 mL |
| Composition | w/v | batch | w/v | batch | w/v | batch | w/v | batch |
| Avacopan | 0.60 | 0.30 | 0.60 | 0.30 | 0.60 | 0.30 | 0.60 | 0.30 |
| Labrofac | | | | | up to | to 50 mL | up to | to 50 mL |
| Lipophile WL | | | | | 100% | (47.25 g) | 100% | (47.25 g) |
| 1349 | | | | | | | | |
| Olive Oil | up to | to 50 mL | up to | to 50 mL | | | | |
| | 100% | (47.00 g) | 100% | (47.00 g) | | | | |
| Labrasol ALF | | | | | | | | |
| Propylene | | | | | | | | |
| Gylcol | | | | | | | | |
| BHT - | | | 0.06 | 0.006 | | | 0.06 | 0.006 |
| butylated | | | | | | | | |
| hydroxytoluene | | | | | | | | |
| Total | 100 | 47.30 g | 100 | 47.306 g | 100 | 47.55 g | 100 | 47.556 g |
| | | (50 mL) | | (50 mL | | (50 mL) | | (50 mL) |

TABLE 2
Compositions for Avacopan Solution Formulations 6 mg/mL and 12 mg/mL

| | 4A | | 4B | | 5A | | 5B | |
|---|---|---|---|---|---|---|---|---|
| | | g/per | | g/per | | g/per | | g/per |
| | | 50 mL | | 50 mL | | 50 mL | | 50 mL |
| Composition | % w/v | batch | % w/v | batch | % w/v | batch | % w/v | batch |
| Avacopan | 2.10 | 1.05 | 2.10 | 1.05 | 1.20 | 0.60 | 1.20 | 0.60 |
| Labrofac | | | | | up to | to 50 mL | up to | to 50 mL |
| Lipophile WL | | | | | 100% | (47.25 g) | 100% | (47.25 g) |
| 1349 | | | | | | | | |
| Olive Oil | | | | | | | | |
| Labrasol ALF | 36.40 | 18.20 | 36.40 | 18.20 | | | | |
| Propylene | 73.00 | to 50 mL | 73.00 | to 50 mL | | | | |
| Gylcol | | | | | | | | |
| BHT - | | | 0.06 | 0.006 | | | 0.06 | 0.006 |
| butylated | | | | | | | | |
| hydroxytoluene | | | | | | | | |
| Total | 100 | 50 mL | 100 | 50 mL | 100 | 47.85 g | 100 | 47.856 g |
| | | | | | | (50 mL) | | (50 mL) |

To the samples of all four formulations marked with a B suffix 6 mg of butylated hydroxytoluene (BHT) was added and stirred until dissolved.

Formulations 5A and 5B had difficulty in dissolving avacopan initially. When avacopan was added gradually to the Labrafac, a solution was obtained.

Samples from each formulation were stored at 2-8° C., 15-25° C. and 45° C. to assess after 7 days.

### RESULTS

Avacopan in the olive oil stress (1A and 1B) data failed with only 51% assay at 7 days at 45° C. with or without BHT. This significant drop corresponds to the solidified particles seen for these samples with or without BHT.

Avacopan in Labrafac (6 mg/mL) with and without BHT (2A and 2B) stress data up to 7 days show no significant impact on assay and impurities level at all conditions tested. The solution appeared clear with no visible particles for all samples.

Avacopan in Labrafac (12 mg/mL) with and without BHT (5A and 5B) stress data at 7 days show no significant impact of condition on assay. However, there is a slight increase in total impurities at 15-25° C. and 45° C. when compared to the 2-8° C. data. The solution appeared clear with no visible particles for the 2-8° C. sample, however a few particles were seen in the 15-25° C. and 45° C. samples.

Avacopan in Labrasol ALF+Propylene Glycol (21 mg/mL) with and without BHT (4A and 4B)+PG+BHT, 21.00 mg/mL) resulted in avacopan separating out of solution overnight.

Numerous formulations were considered for this oral solution varying the avacopan and excipient concentrations. The best vehicle was determined to be Labrasol.

The following excipients were eliminated from use upon solubility and pediatric considerations:
Labrafil M 1944 CS (Oleoyl polyoxyl-6 glycerides)·
Lauroglycol 90 (Propylene glycol monolaurate (type II)
Macrogols E400 (Lutrol E400)·
Transcutol HP (Diethylene glycol monoethyl ether)·
Polysorbate 80
Polysorbate 20
Cremophor RH40
Soybean oil
Butylated hydroxyanisole

The following excipients were eliminated from use after formulation assessments.
Vitamin E TPGS
PEG 400
Propylene Glycol
Citric acid
Capryol 90
Degee LQ
Lauroglycol 90
Labrasol ALF
Olive oil.

### Example 3-Oral Formulation Development for Pediatric Patients

Avacopan 10 mg capsules were approved by FDA in October 2021 with orphan drug designation, exempting from pediatric drug development. The 10 mg commercial capsules are not suitable for younger pediatric patients due to difficulty in swallowing. Initially, a 6 mg/mL formulation of avacopan in Labrafac™ was prepared for testing. However, the thermodynamic solubility of avacopan in Labrafac™ was determined to be 3.6 mg/mL to 3.8 mg/mL at 3° C. to 25° C., indicating that the 6 mg/mL formulation is supersaturated which could lead to precipitation of the drug substance (avacopan) as a crystalline material, and this was confirmed by solubility and precipitation studies. The physical stability of the 6 mg/mL formulation was determined to at risk of drug substance precipitation over long-term storage. A lower avacopan concentration of 3 mg/mL was made as a consequence to eliminate this risk. The decrease in active concentration was compensated by increasing the level of the vehicle, Labrafac™. Use of spray-dried, amorphous DS is proposed for the 3 mg/mL formulation to aid in the faster dissolution in Labrafac™.

Because the formulation of this disclosure is non-aqueous, it does not require any preservatives, such as antimicrobials, to prevent microbial growth and is therefore more stable for this reason.

### Example 4-Stability Studies

Stability of an avacopan 3 mg/mL oral solution was supported with lot manufactured at 4 L scale. The bulk oral solution was packaged into 123 mL Type III amber glass bottles with a fill volume of 60 mL and 24 mm polypropylene child resistant closure. This is considered the worst-case condition as the head space (51%) is larger than the head space (13%) of the pivotal presentation. The filled bottles were placed on stability at 25° C./60% RH (long term storage), 40° C./75% RH (accelerated storage) and 5° C. (refrigerator).

Release (t=0) data and 6-month stability data under these storage conditions of the lot are summarized in Table 1. All release results met acceptance criteria. The six months stability data under both long term and accelerated storage conditions also met the acceptance criteria with no significant changes in appearance, assay, aniline impurity and total impurity profile (Table 3). The current stability data support room temperature storage and a shelf-life of 12 months.

TABLE 3
Release/stability Results Avacopan Oral Formulation, 3 mg/mL

| Stability Condition | | | 40° C./75% RH | | | 25° C./60% RH | | | 5° C. |
|---|---|---|---|---|---|---|---|---|---|
| | Acceptance | | T = 1 | T = 3 | T = 6 | T = 1 | T = 3 | T = 6 | T = 3 |
| Time point | Criteria | T = 0 | Month | months | Months | month | months | months | months |
| Appearance | Clear, | Conform | Conform | Conform | Conform | Conform | Conform | Conform | Conform |
| | colorless to | | | | | | | | |
| | light | | | | | | | | |
| | yellow | | | | | | | | |
| | solution | | | | | | | | |
| | practically | | | | | | | | |
| | free | | | | | | | | |
| | of particles | | | | | | | | |
| Assay (% | 90.0-110.0 | 98.9 | 99.6 | 98.6 | 98.9 | 98.9 | 99.1 | 98.9 | 99.1 |
| Label | | | | | | | | | |
| Claim) | | | | | | | | | |
| Aniline | ≤0.5% | 0.09 | 0.14 | 0.13 | 0.15 | 0.14 | 0.13 | 0.14 | 0.14 |
| Any single | ≤0.2% | Not | Not | Not | Not | Not | Not | Not | Not |
| unspecified | | Detected | Detected | detected | Detected | detected | detected | detected | detected |
| impurity | | | | | | | | | |
| Total | ≤3.0 | 0.09 | 0.14 | 0.13 | 0.15 | 0.14 | 0.13 | 0.14 | 0.14 |
| Impurities | | | | | | | | | |
| (%) | | | | | | | | | |

### Example 5-Administration of Avacopan Liquid Formulation to Pediatric Patients

Pediatric patients will receive twice-daily (BID) administered 3 mg/ml of avacopan in Labrafac™ liquid formulation, or alternatively oral tablets of 10 mg avacopan (Tavneos®) for 52 weeks.

### Doses and Regimens of Oral Liquid Formulation to Pediatric Patients:

Pediatric Patients aged 12 to <18 years (adolescent patients) and weighing at least 50 kg (≥50 kg) at day 1, will be given the option to take either the liquid formulation or the capsule formulation orally with food as 30 mg twice daily.

Pediatric subjects with bodyweight ≥15 kg to <20 kg in the ages 6 to <12 years (children patients) will be administered avacopan 9 mg BID with food as a liquid formulation. Pediatric subjects with bodyweight ≥20 kg to <50 kg in the ages from 6 to <12 years and adolescent subjects ages 12 to <18 years and with bodyweight ≥20 kg to <50 kg will be administered 15 mg BID as liquid formulation with food. Administration of Doses are recommended to be with food approximately 12 hours apart.

Formulation and Dose for Ages 6 to <12 Years

| | 3 mg/mL of avacopan in Labrafac ™ Liquid |
|---|---|
| Body Weight | Formulation |
| ≥15 kg to <20 kg | 9 mg (3 mL) BID |
| ≥20 kg to <50 kg | 15 mg (5 mL) BID |
| ≥50 kg | 30 mg (10 mL) BID |

Formulation and Dose for Ages 12 to <18 Years

| | 3 mg/mL of avacopan in Labrafac ™ Liquid |
|---|---|
| Body Weight | Formulation |
| ≥20 kg to <50 kg | 15 mg (5 mL) BID |
| ≥50 kg | 30 mg (10 mL) BID or |
| | 3 (10 mg) capsules BID of Tavneos ®* |

BID = twice a day.
*Subjects who are 12 to <18 years of age and >50 kg, have the option to choose either 10 mg capsule or liquid formulation

### Inclusion Criteria:

Male and female children and adolescents from 6 to <18 years of age

Clinical diagnosis of granulomatosis with polyangiitis (GPA) or microscopic polyangiitis (MPA), consistent with Chapel-Hill Consensus Conference definitions (Jennette et al, 2013)

Newly diagnosed or relapsed AAV with positive test for anti-PR³ or anti-MPO antibodies

At least 1 PVAS major item, at least 3 PVAS nonmajor items, or at least the 2 renal items of proteinuria and hematuria.

eGFR >15 mL/min/1.73 m2 (using modified Schwartz equation per central lab guidelines)

Participants must have a bodyweight of ≥15 kg at day 1.

### Exclusion Criteria:

Any other known multisystem autoimmune disease including eosinophilic granulomatosis with polyangiitis (Churg-Strauss), systemic lupus erythematosus, IgA vasculitis (Henoch-Schönlein), rheumatoid vasculitis, Sjogren's syndrome, anti-glomerular basement membrane disease, or cryoglobulinemic vasculitis.

Alveolar hemorrhage requiring invasive pulmonary ventilation support anticipated to last beyond the screening period of the study.

Any medical condition requiring or expected to require continued use of immunosuppressive treatments, including corticosteroids that may cause confoundment with study assessments and study conclusions.

### Primary Outcome Measures:

1. At Week 26: Proportion of Participants Achieving Disease Remission at Week 26 According to the Birmingham Vasculitis Activity Score (BVAS)
2. At Week 52: Proportion of Participants with Sustained Disease Remission at Week 52 According to the PVAS

### Secondary Outcome Measures:

1. At Week 26: Proportion of Participants Achieving Disease Remission at Week 26 According to the Pediatric Vasculitis Activities Score (PVAS)

2. At Week 52: Proportion of Participants with Sustained Disease Remission at Week 52 According to the BVAS

3. Up to Week 52: Proportion of Participants With PVAS of 0 Over Time Through Week 52

4. Up to Week 52: Proportion of Participants With BVAS of 0 Over Time Through Week 52

5. Baseline up to Week 52: Change From Baseline Over 52 Weeks in Urinary Albumin-Creatinine Ratio (UACR)

6. Baseline up to Week 52: Change From Baseline Over 52 Weeks in Estimated Glomerular Filtration Rate (eGFR)

7. Baseline up to Week 52: Change From Baseline Over 52 Weeks In Physician Global Assessment (PGA) of Disease Activity

8. Baseline up to Week 52: Change From Baseline Over 52 Weeks in Pediatric Vasculitis Damage Index (PVDI)

9. Screening up to Week 52: Number of Glucocorticoid Dosages Administered

10. Day 1 and Week 2: Proportion of Participants Across the Taste Score Categories of the TASTY Faces Scale.
The TASTY faces scale will be utilized to assess the taste of the oral pediatric formulation. A 7-point version of the scale will be used, where higher scores correspond to faces showing favorable tastes. Upon drug administration, the child will be shown the TASTY scale and asked to rate his/her perception of tastiness by pointing to the appropriate face on the scale.

11. Day 1 and Week 2: Taste and Acceptability Score of Avacopan per TASTY Faces Scale.
The TASTY faces scale will be utilized to assess the taste of the oral pediatric formulation. A 7-point version of the scale will be used, where higher scores correspond to faces showing favorable tastes. Upon drug administration, the child will be shown the TASTY scale and asked to rate his/her perception of tastiness by pointing to the appropriate face on the scale.

Endpoint definitions for Example 5

The term “Remission” refers to:
1. Pediatric Vasculitis Activity Score (PVAS) of 0 at week 26 with prednisone-equivalent dose of ≤0.2 mg/kg/day (max 10 mg/day)

### OR

2. PVAS of 0 at for at least 4 weeks prior to week 26, provided the last PVAS assessment is within the prior 8 weeks, irrespective of the dose of glucocorticoids being received

The term “Sustained remission” refers to: Remission at weeks 26 and 52, with same conditions as above at the week 52 timepoint, without relapses between weeks 26 and

The term “Relapse” refers to: Worsening of disease after previously achieving remission involving one of the following: 1) ≥1 PVAS major item, 2) ≥3 PVAS nonmajor items on a trial visit, or 3) 1 or 2 PVAS nonmajor items for 2 consecutive trial visits

### Inclusion Criteria:

Participant provided informed consent/assent before the initiation of any trial-specific activities/procedures; AND/OR Participant's legally authorized representative provided informed consent before the initiation of any trial-specific activities/procedures when the participant had any kind of condition that, in the opinion of the investigator, might compromise the ability of the participant to give written informed consent; OR Participant's legally authorized representative provided informed consent when the participant was legally too young to provide informed consent, and the participant provided written assent based on local regulations and/or guidelines before the initiation of any trial-specific activities/procedures.
Male and female children and adolescents from 6 to <18 years of age
Clinical diagnosis of GPA or MPA (new or relapsed) consistent with Chapel Hill Consensus Conference definitions
Positive test for anti-proteinase 3 (PR³) or anti-myeloperoxidase (MPO) antibodies
Presentation of at least one major item, at least three nonmajor items, or at least two renal items of proteinuria and haematuria on the PVAS receiving or requiring induction treatment with an RTX or CYC-containing regimen within 3 months prior to day 1. Note: The PVAS was developed and validated based on paediatric-appropriate modification of the BVAS version 3. Major and nonmajor item classifications in the PVAS should follow BVAS principles
eGFR of ≥15 mL/min/1.73 m2
Participants must have a bodyweight of ≥15 kg on day 1

### Exclusion Criteria-Disease Related

Any other known multisystem autoimmune disease including eosinophilic GPA (Churg-Strauss), systemic lupus erythematosus, immunoglobulin A vasculitis (Henoch-Schönlein), rheumatoid vasculitis, Sjögren's disease, anti-glomerular basement membrane disease, or cryoglobulinaemic vasculitis
Dialysis or plasmapheresis within 2 weeks before screening
Have had a kidney transplant

### Exclusion Criteria-Other Medical Conditions

Current or past medical history of malignancy
Alveolar hemorrhage requiring invasive pulmonary ventilation support anticipated to last beyond the screening period of the trial
Any medical condition requiring or expected to require continued use of immunosuppressive treatments, including corticosteroids that may cause confoundment with trial assessments and trial conclusions
Acute or chronic active hepatitis B virus (HBV), hepatitis C virus (HCV), or HIV infection during screening:
   HBV: Hepatitis B surface antigen positive or total hepatitis B core antibody (HBcAb) positive and HBV DNA positive. HBV DNA will be used as a reflex test if total HBcAb is positive. Those with negative HBV DNA may be included in the trial. Hepatitis B surface antibody is not exclusionary
   HCV: HCV RNA positive. HCV RNA will be used as a reflex test if hepatitis C antibody is positive
   HIV: Positive HIV-1/2 immunoassay
Positive test for active or latent tuberculosis (TB) during screening confirmed by
   Positive QuantiFERON or by T-SPOT test
History of non-TB mycobacteria, opportunistic infections (eg, cytomegalovirus, pneumocystis pneumonia, aspergillus, histoplasma, coccidioidomycosis), without appropriate standard course of therapy meeting local guidelines
History of moderate or severe congenital or acquired immunodeficiency
Aspartate aminotransferase, alanine aminotransferase, or alkaline phosphatase >2.0 x the upper limit of normal (ULN). Total bilirubin >1.5 x the ULN. Note: A participant with documented Gilbert's syndrome with total bilirubin <2.0 x ULN may be eligible
Active infection of any kind (including COVID-19 and excluding fungal infections of nail beds) or any major episode of infection requiring hospitalisation or treatment with intravenous anti-infective agents within 4 weeks of signing informed consent or completion of oral anti-infective agents within 2 weeks prior to providing informed consent. Entry into the trial may be reconsidered once the infection has fully resolved.

### Exclusion Criteria-Prior/Concomitant Therapy

Received a strong or moderate inducer of the cytochrome P450 3A4 (CYP3A4) enzyme within 1 week before day 1

### Exclusion Criteria-Prior/Concurrent Clinical Study Experience

Currently receiving treatment with another investigational device or drug study, for <30 days or 5 half-lives of the investigational device or drug study since ending treatment on another investigational device or drug study. Other investigational procedures and participation in observational research studies while participating in this trial are excluded.
Received a live, replication-competent vaccine within 4 weeks before signing the informed consent form
Previously received avacopan without clinical benefit per the investigator's opinion or received avacopan within 8 weeks before signing of the informed consentOther

### Other Exclusions

Female participants of childbearing potential unwilling to use protocol-specified method of contraception during treatment and for at least 2 months after the last dose of avacopan. Note: Male and female participants of childbearing potential should also follow the relevant local product information requirements for contraception for all concomitant medications received in the trial (eg, CYC).
Female participants who are breastfeeding or who plan to breastfeed during treatment and for at least 2 months after the last dose of avacopan. Note: Female participants of childbearing potential should also follow the relevant local product information requirements for breastfeeding for all concomitant medications received in the trial (eg, CYC).
Female participants planning to become pregnant while during treatment and for at least 2 months after the last dose of avacopan. Note: Female participants who are planning to become pregnant while on trial should also follow the relevant local product information requirements for pregnancy for all concomitant medications received in the trial (eg, CYC).
Female participants of childbearing potential with a positive pregnancy test assessed at screening and/or day 1 before dose assignment by a highly sensitive serum or urine pregnancy test, respectively
Any contraindication, including known hypersensitivity to avacopan components or any of the investigational products, or noninvestigational products/auxiliary medicinal products or their components
Participant likely to not be available to complete all protocol-required trial visits or procedures, and/or to comply with all required trial procedures (eg, Clinical Outcome Assessments) to the best of the participant's and investigator's knowledge
History or evidence of any other clinically significant disorder, condition, or disease (except for those outlined above) that-in the opinion of the investigator or physician, if consulted-would pose a risk to participant safety or interfere with the trial evaluation, procedures, or completion.

### Background Immunosuppression

All participants will receive standard of care (SOC) background immunosuppressive therapy, for induction and maintenance, at the discretion of the investigator and as supported by current guidelines, product labels, and local practices and informed by the individual participant's clinical condition, preferences, and values for treatment of pediatric AAV.
Dose modification/reduction or temporary cessation should be managed in accordance with the regional SOC and guidelines.

### Example 6-Avacopan in combination with rituximab (RTX) or cyclophosphamide (CYC) for addressing medical needs of pediatric patients with GPA/MPA

Dosing regimens for alternative primary remission-induced treatment of pediatric AAV-cyclophosphamide (CYC) versus rituximab (RTX)

The Doses and Regimens of the Oral Liquid Formulation of avacopan to Pediatric Patients described in Example 4 can be combined with CYC or RTX dosing regimens. Examples of dosing regimens for CYC and RTX include the following:

A. Alternative intermittent intravenous CYC regimens

Regimen 1 (adapted from the European Alliance of Associations for Rheumatology adult recommendations for AAV)·
15 mg/kg (maximum 1.2 gm) of CYC every 2 weeks for the first 3 doses, then every 3 weeks for the next 3 to 6 doses
   Regimen 2 (adapted from the National Institute of Health Systemic Lupus Erythematosus protocol)·
750 mg/m² every 4 weeks for 4 to 7 doses
   Proposed therapy duration
Minimum of 3 months
Continue beyond 3 months until sustained disease inactivity (PVAS=0, no elevated laboratory markers of inflammation)·
Maximum of 6 months

B. Alternative RTX regimens

Regimen 1 (adapted from initial adult trials)·
375 mg/m²; 4 doses given 1 week apart
   Regimen 2 (most common pediatric practice)·
750 mg/m²; 2 doses given 2 weeks apart

### PRIMARY ENDPOINTS

| OBJECTIVE | ENDPOINT |
|---|---|
| To explore the efficacy of avacopan | Proportion of subjects achieving |
| | disease remission at week 26 |
| | according to the Pediatric Vasculitis |
| | Activities Score (PVAS). |
| | Proportion of subjects with sustained |
| | disease remission at week 52 |
| | according to the PVAS. |
| | Remission defined according to the PVAS |
| | as either: |
| | PVAS score of 0 with |
| | prednisone-equivalent dose of ≤0.2 |
| | mg/kg/day (maximum dosage |
| | of 10 mg/day) within the past |
| | 4 weeks. |
| | OR |
| | PVAS score of 0 for at least |
| | 4 weeks, provided the last PVAS |
| | assessment is within the prior |
| | 8 weeks, irrespective of the dose of |
| | glucocorticoids being received. |
| | Sustained remission defined as: |
| | PVAS-based remission at week 26 and |
| | remission at week 52, without relapses |
| | between the 2 time points. |
| To explore the safety and tolerability of | Treatment-emergent adverse events, vital |
| avacopan | signs, physical examination, and clinical |
| | laboratory findings |

### SECONDARY ENDPOINTS

| OBJECTIVE | ENDPOINT |
|---|---|
| To characterize the pharmacokinetics of | Plasma concentrations of avacopan at |
| avacopan | prespecified timepoints/visits through end |
| | of trial |
| To explore the safety and tolerability of | Treatment-emergent adverse events, vital |
| avacopan | signs, physical examination, and clinical |
| | laboratory findings |
| To explore other efficacy-related outcomes | Proportion of participants achieving |
| | disease remission at week 26 |
| | according to the BVAS |
| | Proportion of participants with |
| | sustained disease remission at week |
| | 52 according to BVAS |
| | Remission defined according to the BVAS |
| | as either |
| | BVAS score of 0 with prednisone- |
| | equivalent dose of ≤0.2 mg/kg/day |
| | (maximum dosage of 10 mg/day) |
| | within the past 4 weeks |
| | OR |
| | BVAS score of 0 for at least 4 |
| | weeks, provided the last BVAS |
| | assessment is within the prior 8 |
| | weeks, irrespective of the dose of |
| | glucocorticoids being received |
| To explore other efficacy-related outcomes | Sustained remission defined as: BVAS- |
| | based remission at week 26 and remission at |
| | week 52, with prednisone-equivalent dose |
| | of ≤0.2 mg/kg/day, without relapses |
| | between the 2 time points |
| | Proportion of participants with |
| | PVAS of 0 over time through week |
| | 52 |
| | Proportion of participants with |
| | BVAS of 0 over time through week |
| | 52 |
| | Changes from baseline over 52 |
| | weeks in urinary albumin-creatinine |
| | ratio (UACR) |
| | Changes from baseline over 52 |
| | weeks in eGFR |
| | Change from baseline over 52 weeks |
| | in Physician global assessment |
| | (PGA) of disease activity |
| | Change from baseline over 52 weeks |
| | in Paediatric Vasculitis Damage |
| | Index (PVDI) |
| | Cumulative glucocorticoid dosages |
| | from screening to week 52 |
| To explore the acceptability and palatability | Mean score of taste and acceptability |
| of the oral liquid pediatric formulation | Number of participants across the |
| | taste score categories |

Although the foregoing disclosure has been described in some detail by way of illustration and example for purposes of clarity of understanding, one of skill in the art will appreciate that certain changes and modifications may be practiced within the scope of the appended claims. Where a conflict exists between the instant application and a reference provided herein, the instant application shall dominate.

What is claimed is:

1. A formulation comprising avacopan, or a pharmaceutically acceptable salt thereof, and one or more medium-chain triglycerides.

2. The formulation of claim 1, wherein the one or more medium-chain triglycerides are triglycerides of saturated fatty acids that are from 6 to 12 carbons in length.

3. The formulation of claim 1, wherein the one or more medium-chain triglycerides comprises triglycerides of caprylic and capric acids.

4. The formulation of claim 3, wherein the one or more medium-chain triglycerides are triglycerides of about 50% to about 80% capric acid and from about 20% to about 50% caprylic acid.

5. The formulation of claim 4, wherein the one or more medium-chain triglycerides are triglycerides of at least about 95% of caprylic and capric acid.

6. The formulation of claim 1, comprising avacopan in an amount of 0.1-1.0% (w/w).

7. The formulation of claim 6, comprising avacopan in an amount of 0.2, 0.3%, 0.4%, 0.5%, or 0.6% (w/w).

8. The formulation of claim 6, comprising avacopan in an amount of 0.3% (w/w).

9. The formulation of claim 1, comprising avacopan in an amount of 3 mg/ml.

10. The formulation of claim 6, comprising avacopan in an amount of 0.6% (w/w).

11. The formulation of claim 1,
comprising avacopan in an amount of 6 mg/ml.

12. The formulation of claim 1, comprising avacopan in an amount of 0.3% (w/w) and one or more medium chain triglycerides in an amount of 99.7% (w/w).

13. The formulation of claim 1, comprising avacopan in an amount of 0.6% (w/w) and one or more medium chain triglycerides in an amount of 99.4% (w/w).

14. The formulation of claim 1, wherein the formulation is suitable for oral administration.

15. The formulation of claim 1, wherein the formulation is suitable for oral administration to patients who have difficulty swallowing or pediatric patients.

16. The formulation of claim 1, wherein the formulation is an immediate release formulation.

17. The formulation of claim 1, wherein the formulation prior to administration is contained in glass or plastic containers with child-resistant closures.

18. The formulation of claim 17, wherein the child-resistant closure comprises polypropylene liners.

19. (canceled)

20. The formulation of claim 1, wherein avacopan is in a free base form.

21. The formulation of claim 1, wherein the formulation is a liquid formulation.

22. The formulation of claim 1, wherein the formulation does not comprise an antimicrobial agent.

23. A method of treating a disease mediated by C5aR in a subject in need of treatment, the method comprising administering to the subject a therapeutically effective amount of the formulation of claim 1.

24. The method of claim 23, wherein the disease mediated by C5aR is anti-neutrophil cytoplasmic autoantibody-associated vasculitis, complement 3 glomerulopathy, hidradenitis suppurativa, or lupus nephritis, or any combination of the foregoing.

25. The method of claim 24, wherein the disease mediated by C5aR is anti-neutrophil cytoplasmic autoantibody-associated vasculitis.

26. The method of claim 1, wherein the subject receives of avacopan in one or more medium-chain triglyceride selected from one of the following doses: 9 mg BID; 15 mg BID; and 30 mg BID.

27. The method of claim 26, wherein the subject receives 3 mg/ml of avacopan in one or more medium-chain triglyceride at a dose of 9 mg BID.

28. The method of claim 26, wherein the subject receives 3 mg/ml of avacopan in one or more medium-chain triglyceride at a dose of 15 mg BID.

29. The method of claim 26, wherein the subject receives 3 mg/ml of avacopan in one or more medium-chain triglyceride at a dose of 30 mg BID.

30. The method of claim 27, wherein the subject is ≥15 kg to <20 kg.

31. The method of claim 28, wherein the subject is ≥20 kg to <50 kg.

32. The method of claim 29, wherein the subject is ≥50 kg.

33. The method of claim 1, wherein the subject is a patient that has difficulty swallowing solids or is a pediatric patient.

34. The method of claim 1, wherein the subject is administered rituximab or cyclophosphamide in combination with the formulation.

35. The method of claim 34, wherein cyclophosphamide is administered to the subject in an amount of 15 mg/kg every 2 weeks for the first 3 doses, then every 3 weeks for the next 3 to 6 doses.

36. The method of claim 34, wherein cyclophosphamide is administered to the subject in an amount of 750 mg/m² every 4 weeks for 4 to 7 doses.

37. The method of claim 34, wherein rituximab is administered to the subject in an amount of 375 mg/m² in 4 doses given one week apart.

38. The method of claim 34, wherein rituximab is administered to the subject in an amount of 750 mg/m² in 2 doses given 2 weeks apart.

39. A process of making a formulation comprising:
providing crystalline or spray-dried amorphous avocapan; and
dissolving the crystalline or spray-dried amorphous avacopan in one or more medium-chain triglycerides, wherein the one or more medium-chain triglyceride are triglycerides of saturated fatty acids that are from 6 to 12 carbons in length.

40. The process of claim 39, wherein the one or more medium-chain triglyceride are triglycerides of caprylic and capric acids.

41. A formulation of avacopan made by the process of claim 39 [or 40].

42. The formulation of claim 41, comprising avacopan in an amount of 0.1-1.0% (w/w).

43. The formulation of claim 42, comprising avacopan in an amount of 0.3% (w/w).

44. The formulation of claim 43, comprising avacopan in an amount of 3 mg/ml.

45. The formulation of claim 44, wherein the formulation is in liquid form.
